SEQUENCE LISTING (7 sequences)

SEQ ID NO: 1 - DNA, 72 bp, Artificial Sequence
  Target Polynucleotide
ctgaagcttaagctgaagcttaagctgaagcttaagctgaagcttaagctgaagcttaagctgaagcttaag

SEQ ID NO: 2 - DNA, 110 bp, Artificial Sequence
  Polynucleotide Substrate
tgccactcaagcctaagtacgaagtggtctgatcctgctgccactcaagcctaggtacgaagtggtctgatcctgctgccactcaagcctaagtacgaagtggtctgatc

SEQ ID NO: 3 - DNA, 114 bp, Artificial Sequence
  Polynucleotide Substrate
ctgcgatcagacctcttcgtacctcggcttaagtggcaatatgatcagacctcttcgtacctaggcttgagtggcaatatgatcagacctcttcgaacctcggcttgagtggca

SEQ ID NO: 4 - DNA, 110 bp, Artificial Sequence
  Polynucleotide Substrate
tgccactcaagccgaggttcgaagaggtctgatcctgctgccactcaagccgaggttcgaagaggtctgatcctgctgccactcaagccgaggttcgaagaggtctgatc

SEQ ID NO: 5 - DNA, 114 bp, Artificial Sequence; identical to SEQ ID NO: 3
  Polynucleotide Substrate

SEQ ID NO: 6 - DNA, 110 bp, Artificial Sequence
  Polynucleotide Substrate
tgccacttaagcctaagttcgaagaggtctgatcctgctgccacttaagcctaggttcgaagaggtctgatcctgctgccacttaagcctaagttcgaagaggtctgatc

SEQ ID NO: 7 - DNA, 113 bp, Artificial Sequence
  Polynucleotide Substrate
ctgcgatcagacctcttcgtacctcggcttaagtggcaatatgatcagacctcttcgtacctaggcttgagtggcaatatgatcagacctcttgaacctcggcttgagtggca

(19) United States
(12) Patent Application Publication
(10) Pub. No.: US 2009/0053699 A1
(43) Pub. Date: Feb. 26, 2009

(54) Method for Preparing Polynucleotides for Analysis

(75) Inventor: Preben Lexow, Oslo (NO)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Lingvitae AS, Oslo (NO)

(21) Appl. No.: 11/817,286

(22) PCT Filed: Mar. 8, 2006
(86) PCT No.: PCT/GB06/00825

§ 371 Date: Aug. 29, 2008

(30) Foreign Application Priority Data
Mar. 8, 2005 (GB) 0504774.1

(51) Int. Cl. C12Q 1/68; C12P 19/34

(52) U.S. Cl. 435/6; 435/91.5

(57) ABSTRACT

A method for analysing a target polynucleotide having distinct units of nucleic acid sequence comprising: (i) forming a first polynucleotide which is a concatemer having multiple repeating target polynucleotide sequences; (ii) forming on the first polynucleotide a second polynucleotide hybridised to a portion of one or more of the target polynucleotides, such that the portion hybridised, or the portion not hybridised, corresponds to a sequence unit on the target, and determining the sequence unit on the target.

### FIELD OF THE INVENTION

This invention relates to methods for modifying polynucleotides to allow analysis of the polynucleotides to be carried out more readily.

### BACKGROUND TO THE INVENTION

Advances in the study of molecules have been led, in part, by improvement in technologies used to characterise the molecules or their biological reactions. In particular, the study of the nucleic acids DNA and RNA has benefited from developing technologies used for sequence analysis and the study of hybridisation events.

WO-A-00/39333 describes a method for sequencing polynucleotides by converting the sequence of a target polynucleotide into a second polynucleotide having a defined sequence and positional information contained therein. The sequence information of the target is said to be “magnified” in the second polynucleotide, allowing greater ease of distinguishing between the individual bases on the target molecule. This is achieved using “magnifying tags”, which are predetermined units of nucleic acid sequence. Each of the bases adenine, cytosine, guanine and thymine on the target molecule is represented by an individual magnifying tag, converting the original target sequence into a magnified sequence. Conventional techniques may then be used to determine the order of the magnifying tags, and thereby determine the specific sequence on the target polynucleotide.

In a preferred sequencing method, each magnifying tag comprises a label, e.g. a fluorescent label, which may then be identified and used to characterise the magnifying tag.

WO-A-04/094664 describes an adaptation of the conversion method disclosed in WO-A-00/39333. In both methods, it is preferred that each magnifying tag comprises two units of distinct sequence which can be used as a binary system, with one unit representing “0” and the other representing “1”. Each base on the target is characterised by a combination of the two units, for example adenine may be represented by “0”+“0”, cytosine by “0”+“1”, guanine by “1”+“0” and thymine by “1”+“1”.

One difficulty with the prior art methods is that the eventual read-out step is often hindered by the need to discriminate between the different magnifying tags or units. It is therefore desirable to identify improvements which permit discrimination to occur.

### SUMMARY OF THE INVENTION

The present invention provides a method for analysing polynucleotides, preferably those polynucleotides which have been formed with distinct units of polynucleotide sequence each representing a particular characteristic. The method utilises a concatemer of the target polynucleotide, i.e. repeating the sequence of the target polynucleotide, and then forming a further polynucleotide on this, the further polynucleotide being hybridised at specific portions of the target, such that hybridised or non-hybridised sequences can be identified and the order of hybridisation (or non-hybridisation) reveals the identity and/or order of the units of the target polynucleotide. The intention is, preferably, to identify sequentially one unit of each target polynucleotide on the concatemer. In this way, the units to be identified are more separated than if the units of the original target polynucleotide were to be sequenced. Increasing the separation allows the eventual read-out technology to discriminate between the units, thereby improving the efficiency of the eventual sequencing/identification step. Alternatively the method may be carried out so that repeated target polynucleotides are separated by additional nucleic acid sequences which act to space apart the targets, so that analysis can be performed.

According to a first aspect of the present invention, a method for analysing a target polynucleotide having distinct units of nucleic acid sequence, comprises:

(i) forming a first polynucleotide which is a concatemer having multiple repeating target polynucleotide sequences;

(ii) hybridising a portion of the first polynucleotide to a second polynucleotide, such that the portion hybridised, or the portion not hybridised, corresponds to at least a sequence unit on the target, and determining the sequence unit on the target.

In a second aspect of the invention, the concatemer is formed directly on to a second polynucleotide having a predetermined order of defined first and second nucleic acid sequence units. The second polynucleotide is designed to hybridise to specific sequence units on the target. A specific interaction between the concatemer and the second polynucleotide occurs such that there are hybridised portions that can be interrogated to reveal the identity of the sequence unit.

According to a second aspect of the invention, a method for the conversion of a target polynucleotide having distinct units of nucleic acid sequence into a polynucleotide having nucleic acid sequences separating one or more of the distinct nucleic acid sequence units, comprises:

(i) forming a second polynucleotide having defined first and second sequence units in a predetermined order;

(ii) forming on the second polynucleotide, a first polynucleotide made up of a series of the target polynucleotides, wherein the order of the first and second sequence units permits the specific interaction between the first and second polynucleotides, such that distinct units of nucleic acid sequence on the first polynucleotide can be distinguished from other distinct units by the extent of interaction; and optionally

(iii) determining the sequence and/or order of the distinct sequence units on the basis of the interaction to thereby determine one or more specific sequence units on the target polynucleotide.

According to a third aspect of the present invention, a method for the formation of a double-stranded polynucleotide, comprises:

(i) forming a first polynucleotide;

(ii) carrying out a rolling circle amplification reaction from a primer molecule attached to the first polynucleotide, wherein the amplification reaction utilises a circular polynucleotide molecule having a sequence that is complementary to repeating units of the first polynucleotide.

### BRIEF DESCRIPTION OF THE DRAWINGS

The invention is described with reference to the accompanying drawings, wherein:

FIG. 1 (a) illustrates the use of different nucleic acid sequences to represent either a “0” or “1” characteristic, and the sequence on the second polynucleotide which will hybridise to the sequence, or not;

FIG. 1 (b) is a graphic illustration of roll back PCT;

FIG. 2 illustrates the masking or unmasking of a target polynucleotide;

FIG. 3 illustrates the use of padlock probes in the interrogation of a nucleic acid sequence which is “unmasked”;

FIG. 4 illustrates the design of a target polynucleotide and the second polynucleotide;

FIG. 5 illustrates the formation of a target polynucleotide having defined units of nucleic acid sequence;

FIG. 6 illustrates the concatemerisation of a target with additional intervening nucleic acid sequence;

FIG. 7 (a) illustrates the use of an array of second polynucleotides used to capture and characterise the target polynucleotide;

FIG. 7 (b) illustrates the use of padlock probes to interrogate the target polynucleotide;

FIG. 8 illustrates the hybridisation between the concatemerised target polynucleotides and the second polynucleotide and the presence of mismatched (unmasked) sequence;

FIG. 9 illustrates the use of padlock probes to hybridise the unmasked regions of the concatemerised target polynucleotide;

FIGS. 10 to 12 each illustrate the use of restriction enzyme substrate sequences to interrogate the target polynucleotide; and

FIG. 13 is an electrophoresis gel showing the products of restriction enzyme digestion of the hybrids shown in FIGS. 10 to 12.

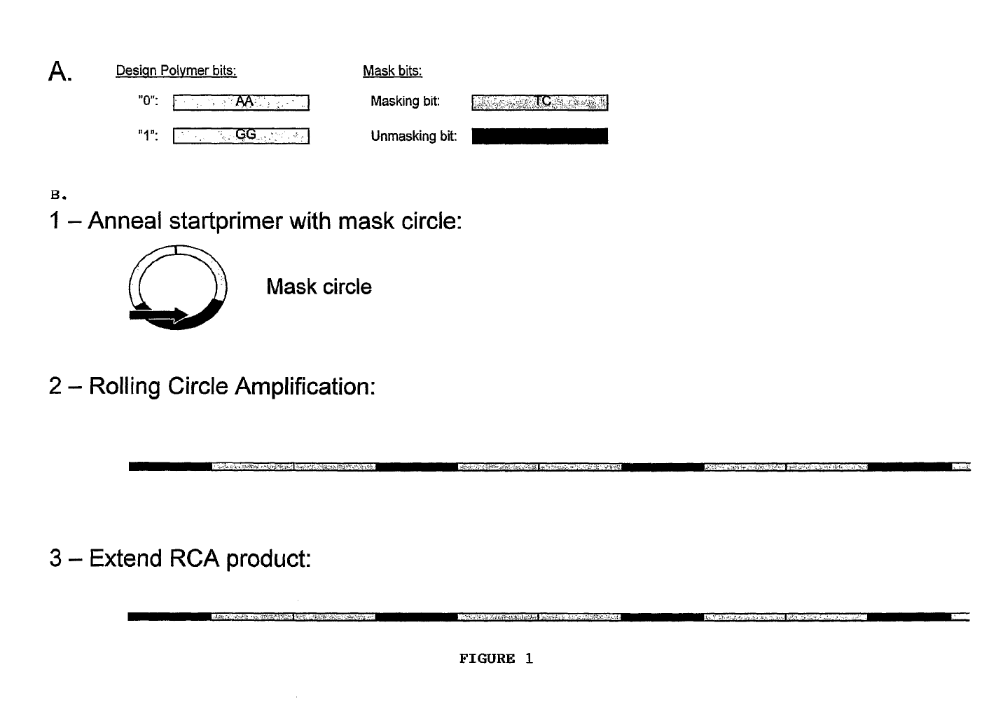

### DESCRIPTION OF THE INVENTION

The term “polynucleotide” is well known in the art and is used to refer to a series of linked nucleic acid molecules, e.g. DNA or RNA. Nucleic acid mimics, e.g. PNA, LNA (locked nucleic acid) and 2′-O-methRNA are also within the scope of the invention.

The reference herein to the bases A, T(U), G and C, relate to the nucleotide bases adenine, thymine (uracil), guanine and cytosine, as will be appreciated in the art. Uracil replaces thymine when the polynucleotide is RNA, or it can be introduced into DNA using dUTP, again as well understood in the art.

The term “first polynucleotide” is used herein to refer to the concatemerised target polynucleotide, i.e. the first polynucleotide comprises repeated target polynucleotide sequences. The target polynucleotides may be linked sequentially, or there may be additional nucleic acid sequences separating the target polynucleotides.

The term “second polynucleotide” is used herein to refer to a polynucleotide intended to hybridise to regions of the first polynucleotide. The second polynucleotide may also be referred to as a masking polynucleotide as it acts to prevent interrogation of those regions of the first polynucleotide to which it hybridises. The regions of the first polynucleotide that are not hybridised are said to be “unmasked”.

The method of the present invention is used to convert a target polynucleotide having distinct units of nucleic acid sequence into a polynucleotide where the distinct units of nucleic acid sequence can be interrogated at intervals more spaced apart then that of the target. This has the benefit of separating the units to permit the ultimate read-out steps to be performed with more accuracy and discrimination. The invention relies on the formation of a concatemer of the target polynucleotide which permits subsequent interrogation to be performed on selected units; the interrogated units are representative of the distinct units of the original target polynucleotide.

Having formed the concatemer, distinct units of the target polynucleotide can be interrogated in various ways to reveal the identity of one or more of the units or to identify the presence of a specific sequence present on the target.

The preferred way of interrogating the concatemer is to hybridise the concatemer with one or more second polynucleotides of defined sequence, such that the second polynucleotide acts to mask portions of the concatemer, leaving the unmasked portions (sequence units) available for interrogation with, for example, a labelled polynucleotide specific for that portion. Identification of the labelled polynucleotide reveals the identity of the sequence units. Different sequence units on each of the concatemer's target polynucleotides can be identified in this way.

The method of the invention relies on the use of a target polynucleotide that is comprised of defined nucleic acid sequence “units”, where each unit represents a specific characteristic of an earlier molecule. For example, each unit may represent a specific base on an original polynucleotide molecule of unknown sequence. Each unit will preferably comprise 2 or more nucleotide bases, preferably from 2 to 50 bases, more preferably 5 to 20 bases and most preferably 5 to 10 bases, e.g. 6 bases. There are preferably at least two different bases contained in each unit. The design of the units is such that it will be possible to distinguish the different units during a “read-out” step, e.g. involving the incorporation of detectably labelled nucleotides in a polymerisation reaction, or on hybridisation of complementary oligonucleotides. Methods in which a molecule is converted into a target polynucleotide are well known in the art, for example as described in WO-A-00/39333 and WO-A-04/094664 the content of each being incorporated herein by reference. However, a unit may be a single base, if necessary.

In a preferred embodiment the target polynucleotide comprises a series of distinct units, the combination of which imparts sequence or other information. For example, two units may be used as a binary system, with one unit representing “0” and the other representing “1”. The combination of different units may represent bases on an original polynucleotide that is being sequenced. This is disclosed in WO-A-04/094664.

The concatemer therefore comprises repeated units of nucleic acid sequence.

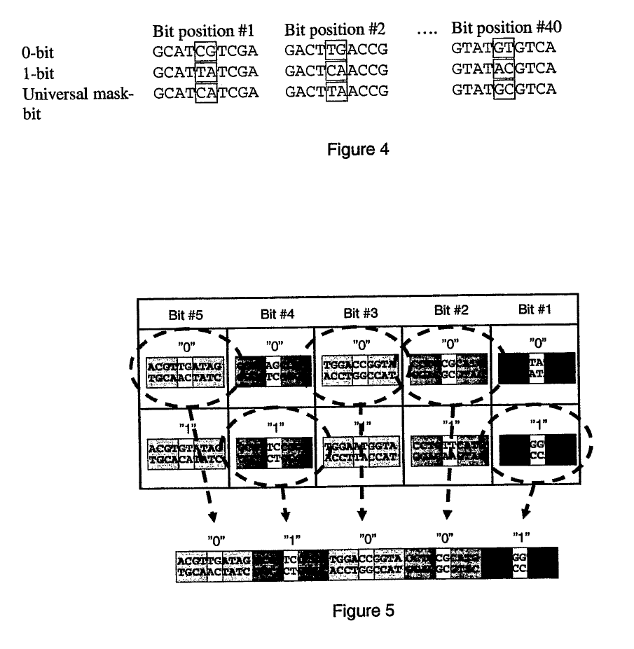

The target polynucleotide may be designed so that a part of each “unit” represents the characteristic of the original molecule under study, but that each unit also comprises nucleic acid sequence of known sequence. The known sequence can be different for each unit, so that, prior to analysis, at least a partial sequence of the target is known. This allows the second polynucleotide to be designed for hybridisation with the target. This is shown in more detail in FIGS. 4 and 5, where there are two possible unit sequences for each unit position on the target; each unit may represent “0” or “1”, depending on the sequence of the two nucleotides. However, the remaining eight nucleotides are the same for “0” or “1”, for each position, but different to the next unit position. In this way, the eventual interrogation can be designed so that hybridisation with specific units can be performed leaving units at specific positions un-hybridised, thereby allowing interrogation of the un-hybridised units to take place. In one embodiment, the intention is to interrogate a different unit position on each target polynucleotide of the concatemer, thereby allowing further separation of the units and improving the ability of the eventual read-out step to discriminate between the sequence units.

Alternatively, if the target sequence is not known, the target polynucleotide may be ligated to a known sequence prior to concatemerisation, so that the concatemer comprises both target polynucleotide sequences and known sequences, so that hybridisation can occur between the concatemer and the second polynucleotide. In this embodiment, the known sequences should be of sufficient length to permit hybridisation with the second polynucleotide to occur. For example, the known sequences should be more than 100 nucleotides, preferably more than 500 nucleotides. This provides separation between the hybridised sequences and the un-hybridised (target) sequences, which can then be interrogated.

The conditions necessary for carrying out the method of the invention, including temperature, pH, buffer compositions etc., will be apparent to those skilled in the art.

The method of the invention follows three essential steps:

i. concatemerisation of the target polynucleotide;

ii hybridising the concatemer with a further (second) polynucleotide of defined known sequence;

ii identifying the units on the concatemer that are hybridised or, preferably, that are not hybridised.

The concatemer may be formed in any suitable way. In a preferred embodiment, the target polynucleotide is circularised and the polymerase reaction is carried out to form the concatemer.

### Circularisation

The target may be circularised in any convenient way. In one embodiment, the single-stranded target is hybridized to the 3′ end of the second polynucleotide. Both the 5′ and the 3′ end of the target molecule will hybridize to the second polynucleotide and will be ligated together forming a single-stranded circle. The efficiency of circle ligations is much better with increased complementarity and it is preferred to use at least 6 complementary nucleotides, preferably at least 9 complementary nucleotides for hybridisation to the second polynucleotide. The ligase can be any available ligase, but is preferably T4 DNA ligase, E. coli DNA ligase or Taq DNA ligase.

In an alternative method, a support oligonucleotide can be used to hybridise to the target and to ligate to the second polynucleotide. In one embodiment of this, the hybrid forms a partially double-stranded molecule with an overhang complementary to the second polynucleotide's 3′ end. The support oligonucleotide can then be ligated to the second polynucleotide at the 3′ end. The 5′ end of the target is also complementary to the second polynucleotide and so the target will hybridise to the second polynucleotide bringing the two ends of the target into position for a ligase to join the two ends of the target, forming a circle. The support oligonucleotide acts to help retain the now circularised target at the second polynucleotide, ready for concatemerisation.

In an alternative embodiment, the support oligonucleotide is used as a splint oligonucleotide. The splint oligonucleotide is complementary to the 3′ and 5′ regions of the target and brings these two ends into proximity, allowing a ligase reaction to occur. The hybrid of the now circularised target and splint oligo is then brought into contact with the second polynucleotide and the target hybridises at the end of the second polynucleotide in a way designed to bring the splint oligonucleotide into proximity to the second polynucleotide. Ligation of the splint oligo to the second polynucleotide can then occur, which aids future polymerase amplification by providing a sufficient number of bases to which the circular target can hybridise to prior to a further round of amplification.

The support oligonucleotide will be of a size sufficient to aid hybridisation and circularisation with the target.

In a third alternative method, a splint oligo is used. The splint oligo is a short single-stranded molecule complementary to the 3′ and 5′ ends of the target. Hence, the splint oligo will bring the termini of the target into position for a ligase to join the two ends, forming a circle. The splint can then be removed by using exonucleases e.g. exonuclease I and exonuclease III, to digest the splint oligo.

### Concatemerisation

The target polynucleotide can be concatemerised using a polymerase reaction. In one embodiment, the circularised target polynucleotide acts as a template for a polymerase reaction. As the template is a circular molecule, the technique used is commonly known as Rolling Circle Amplification (RCA). Several variants of this method exist, as reviewed in Richardson et al., Genetic engineering, 25, 51-63. Linear RCA utilises one primer producing one concatemer from each template. Exponential RCA utilises two primers, where one is complementary to the target to be amplified, while the other is complementary to the product generated by the first primer. Hence, the second primer initiates the synthesis of multiple concatemerised copies from one target polynucleotide. Multiply-primed RCA utilises a set of random hexamers as primers. These primers initiate the synthesis of multiple concatemerised copies from one target polynucleotide. Secondary non-specific priming events can occur subsequently on the displaced product strands of the initial RCA step. The polymerase activity can be initiated at the 3′ end of the secondary polynucleotide. Several polymerases can be used, including Sequenase, Bst DNA polymerase (large fragment), Klenow exo-DNA polymerase, which are all polymerases operating at 37° C. and displaying the crucial strand displacement ability necessary for making the concatemers. Also, the heat-stable Vent exo-DNA polymerase may be used. However, the enzyme shown in the literature to be most efficient on acting on circular templates is phi29 polymerase, and this is preferred.

Alternatively, concatemerisation takes place by ligating multiple copies of the target molecule to form one continuous single stranded molecule. In this way a circularised target polynucleotide is unnecessary.

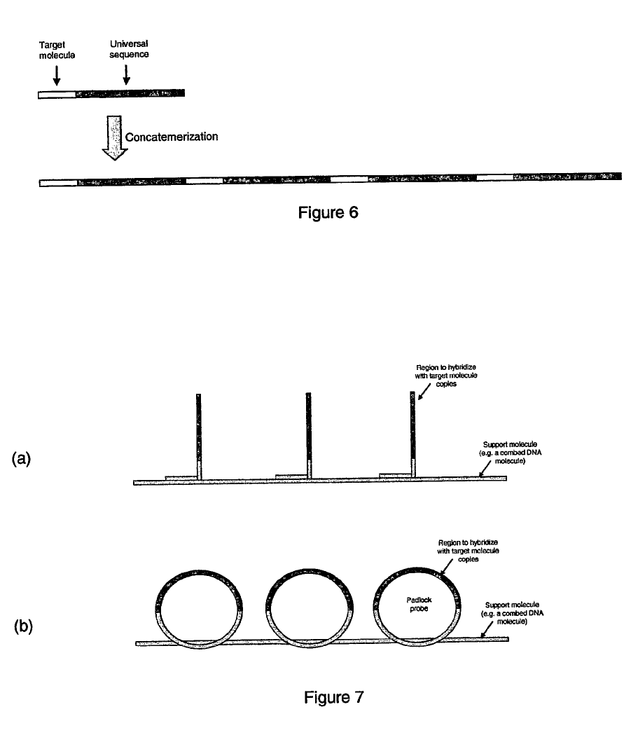

Intervening nucleic acid sequences may also be present as shown in FIG. 6. This is often desirable as the intervening nucleic acid can be of known sequence which will aid the hybridisation to the second polynucleotide.

Hybridisation with Mask (Second) Polynucleotide

The hybridisation to the second polynucleotide can be carried out directly as the concatemer is produced or a single-stranded blocking molecule can be used and removed with an exonuclease after the concatemerisation has been fulfilled. Accordingly, the second polynucleotide can be present during the formation of the concatemer. This is explained below.

The hybridisation of the concatemer with the second polynucleotide can occur simultaneously as the polymerase reaction proceeds on the concatemer. In this embodiment, the circular target may be attached (ligated) to the second polynucleotide as described above, so that the polymerase product is formed in proximity to the second polynucleotide, aiding hybridisation.

In an alternative method, the hybridisation can also be separated from the concatemerisation reaction by “blocking” the second polynucleotide using a complementary molecule. The blocking molecule can either be synthesised in a separate reaction and then annealed to the second polynucleotide prior to the concatemerisation reaction. Alternatively, the blocking molecule can be synthesised by a polymerase directly on the second polynucleotide by using a short primer. After the concatemerisation reaction, the blocking molecule can be removed using an exonuclease, and the second polynucleotide is then available for hybridisation to the concatemerised target molecule and its polymerised product.

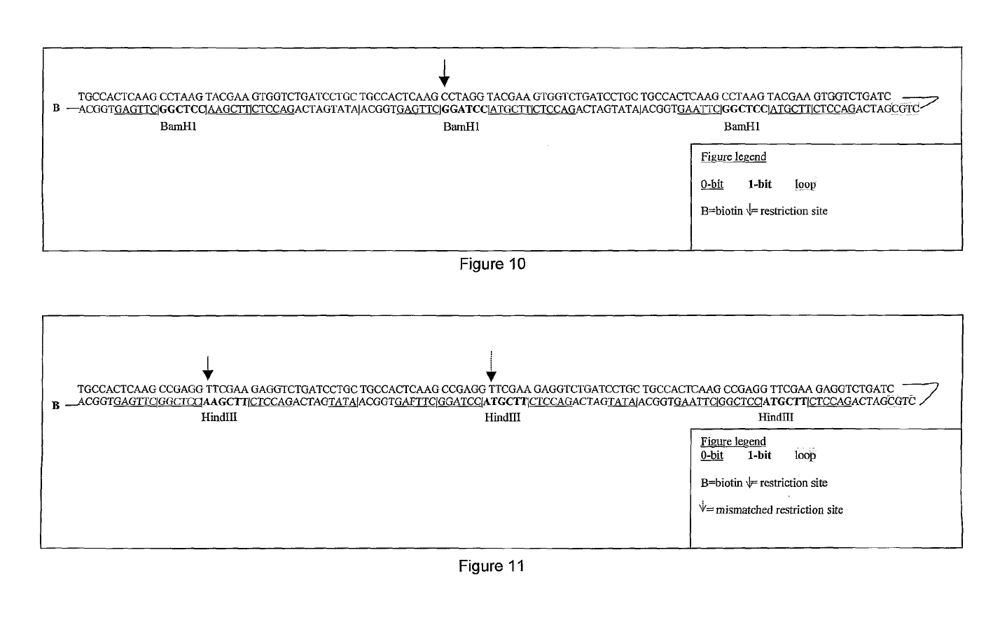

The second polynucleotide will have at least partial complementarity to the concatemer. This is achieved either by knowledge of the partial sequence of the units of sequence on the target, or by incorporating complementary sequences into the formed concatemer. The intention is to hybridise the target to the second polynucleotide, such that there are non-hybridised portions which can be interrogated. Alternatively, the method may be carried out by analysing the hybridised portion. For example, the method may be designed such that restriction enzyme substrate sites are formed if perfect hybridisation occurs at selected regions of the second polynucleotide, and treating the duplex with a restriction enzyme. Monitoring any cleavage product will reveal whether the complementary sequence was present on the target, thereby revealing a characteristic of the original molecule. This is shown in FIGS. 10 to 13.

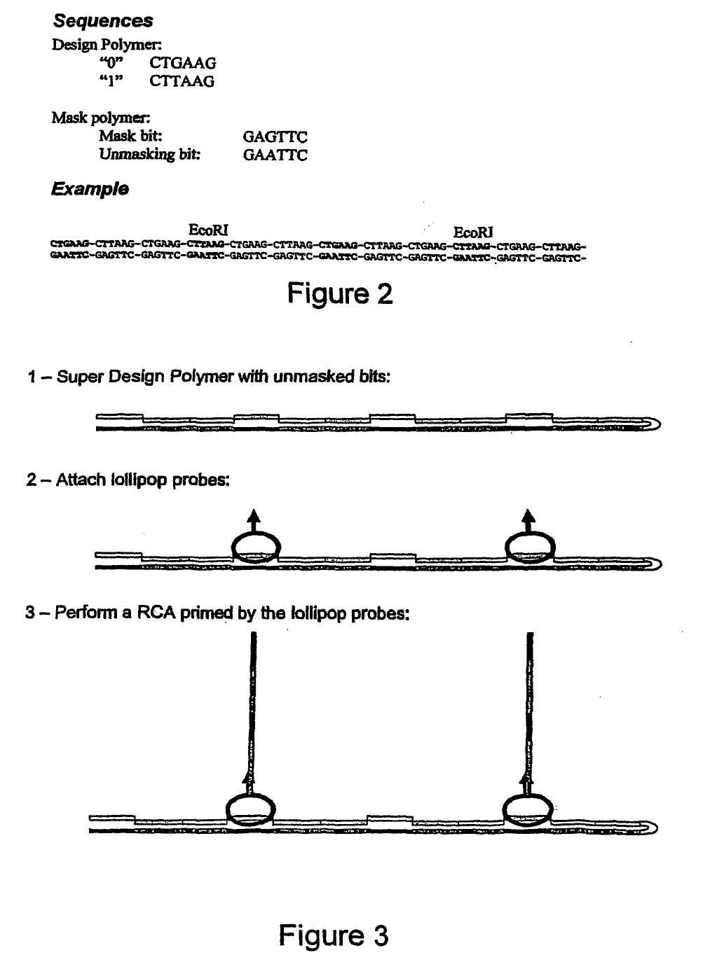

Preferably, the second polynucleotide is said to comprise first and second sequence units in a predetermined order. The intention is to use either or both of the first and second sequence units to mask sequences on the first polynucleotide, to permit selective interrogation of the first polynucleotide to occur. In one embodiment, the second polynucleotide is used to mask selected units on the first polynucleotide to thereby separate units on the first polynucleotide and permit interrogation to occur more readily. The “unmasked” units of the first polynucleotide may be in an order that represents the order of units on the original target polynucleotide, or may be used to create a new order of units which represents specific sequences on the target polynucleotide. For example, with reference to FIG. 2, the interaction between the first and second polynucleotides is carried out to reveal restriction enzyme sites occurring between perfectly complementary sequences on the first and second polynucleotides. Although hybridisation occurs between non-complementary portions (see sequence in bold), this will not permit a restriction enzyme digestion to occur.

If perfect hybridisation occurs, the restriction enzyme substrate can be cleaved. If one or more mismatches occurs, then there will be no cleavage. some restriction enzymes are able to cleave substrates when one or two mismatches are present. If these enzymes are to be used to characterise the target polynucleotide it is preferred that the second polynucleotide is designed so that two or more mismatches are present.

Alternatively, as shown in FIG. 3, the interaction between the first and second polynucleotides results in non-hybridised units which can be interrogated by hybridisation with a subsequent oligonucleotide. This is explained in more detail below.

Multiple second polynucleotides may also be used in an addressable array, wherein the concatemer hybridised to several second polynucleotides with subsequent interrogation permitting identification of hybridisation events. This is shown in FIG. 7 and is explained in more detail below.

### Analysing the Target Polynucleotide

As explained above, the target polynucleotide comprises nucleic acid sequence ‘units’, which represent particular characteristics (e.g. sequence information on an original molecule. The method of the invention is used to identify the units, to thereby determine the characteristics of the original molecule. The concatemer is used to identify the units at intervals more spaced apart than could be achieved if analysis was performed on the single target polynucleotide. In order to carry this out, it is preferred to ‘mask’ selectively units on each copy of the target polynucleotide on the concatemer, with the unmasked units being interrogated and identified.

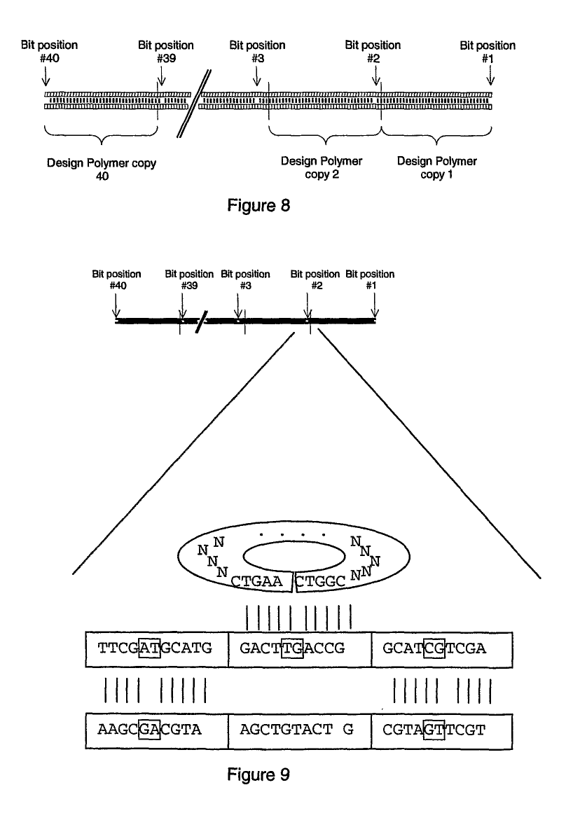

Masking is achieved by use of a second polynucleotide which will mask (hybridize with) most of each target polynucleotide (address) on the first polynucleotide and then let each address unmask one specific bit position. This is shown in FIG. 8. Address 1 will unmask bit position 1, and so on. The procedure for revealing the content of each bit position can then be to ligate the bit with a padlock-probe with, for example a signal moiety, perform a polymerase fill-in reaction with a labeled nucleotide that can be detected. When all of the bit positions are labeled appropriately, the read-out step can be performed to characterise each bit. This procedure is described in more detail below.

### Step 1

In this example, the target polynucleotide comprises at least 40 “bits” of nucleic acid sequence coding for the native (original) DNA sequence to be analysed. Each bit position can have either of two values: 0 or 1. The two bits possible for each position are at least 10 bp long where two bases are unique. These two bases can be positioned anywhere in the bit coding sequence and determine the value of the bit. The remaining bases (at least 8) are preferably identical for the two bit-values at each position, but different for each bit position as illustrated in FIG. 4.

The mask molecule (second polynucleotide) is a single-stranded molecule containing at least 40 copies of the target polynucleotide or complementary sequence. As each target polynucleotide is different with respect to bit value sequence, the mask (second polynucleotide) does not contain 0 or 1 bit sequences, but “universal bits” differing in one base-pair from the value-coding bits as illustrated in FIG. 4.

After the target polynucleotide is concatemerised, the polymerase reaction is carried out.

### Step 2

The resulting hybrid between the mask and the concatemer consists of double-stranded DNA with one mismatch in every bit sequence. However, due to the design of the mask, bit-position one in target polynucleotide copy one will not hybridise, but leave the bit sequence exposed. Further, bit-position two in target polynucleotide copy two will also not hybridise, exposing the sequence of the bit in position two. This mechanism is used to reveal the sequences of all the bits in the original target polynucleotide as illustrated in FIG. 8. Also, the revealed bit values are physically separated by one target polynucleotide length, magnifying the signal chain at least 40-fold. The second polynucleotide may be designed so that there is no hybridisation in the bit (unit) positions to be interrogated. So, for example, in bit position 1 in target 1 the sequence on the second polynucleotide may not have any complementary regions. Similarly, in bit position 2 in target 2 there may be no complementary sequences. This helps separate the bit positions to be interrogated.

### Step 3

Interrogation of the resulting hybrid may be carried out using any convenient read-out technique. In one embodiment it is preferred to use padlock technology (Nilsson et al., Science, 1994; 265 (5181):2085-8 and Baner et al., Curr. Opin. Biotechnol., 2001; 12 (1):11-15). A padlock probe is an oligonucleotide that becomes circularised by DNA ligation in the presence of an appropriate polynucleotide sequence. The reaction requires the two ends of the oligonucleotide to hybridise to adjacent nucleotides on the target for ligation to occur; it is therefore highly specific. As the value of each bit is coded for by two adjacent basepairs these two basepairs can be utilised to perform sequence-specific ligation of padlock probes to the unmasked bits as illustrated in FIG. 9. If bit position one codes for the value 1 only the 1-specific probe will be able to hybridise its two termini bases and be available for a ligation reaction. If a O-specific probe is hybridised to a 1-bit, the padlock probe will not be available for a ligation reaction, and hence it can be washed away. When all the bit-positions have been connected to its corresponding padlock probe, the resulting molecule can be read by standard image acquiring systems provided that the 0-specific and 1-specific padlock probes are labelled with 0- or 1-specific fluorophores.

Once the conversion method has been performed a “read-out step” may be carried out to obtain the sequence information encoded within.

The read-out step may be performed using any suitable technique, for example as described in WO-A-00/39333 and WO-A-04/094663.

One read-out strategy is to use short detectably-labelled oligonucleotides to hybridise to the units on the converted polynucleotide (first or second polynucleotide) and to detect any hybridisation event. The short oligonucleotides have a sequence complementary to specific units of the converted polynucleotide. This is shown in FIG. 7 (a).

The following example illustrates the invention.

### EXAMPLE

In order to demonstrate the “roll-back” principle a 114 nt single-stranded molecule was used as a second polynucleotide substrate and a 38 bp circular target molecule. The substrate molecule was immobilized on 1 μM streptavidin coated paramagnetic beads using biotin to “anchor” the second polynucleotide.

The target molecule is hybridised to the second polynucleotide and phi29 DNA polymerase is added. The polymerase performs an extension using the target as a template. The extended strand is complementary to the second polynucleotide, and will, according to the roll-back theory, hybridise to the second polynucleotide forming an 114 bp double-stranded molecule. Depending on the sequence of the target, the double stranded molecule will contain recognition sites for certain restriction endonucleases (FIGS. 10, 11 and 12):

As shown in FIG. 10, the target with the unit sequence 0100 creates a recognition site for BamH1 in the 2^{nd} bit position in the 2^{nd} target polynucleotide copy. The 2^{nd} bit position in copy 1 and 3 are inactivated by two single basepair mismatch hybridizations in the recognition site. Provided that the roll-back has actually occurred, digestion with BamH1 will yield a 64 bp molecule that can be visualized on a gel.

As shown in FIG. 11, the target with the unit sequence 0010 creates a recognition site for HindIII in the 3^{rd} bit position in the 3^{rd} target copy. The 3^{rd} bit position in copy 1 and 2 are inactivated by a single basepair mismatch in the recognition site. Provided that the rollback has actually occurred, digestion with HindIII will yield a 96 bp molecule that can be visualized on a gel.

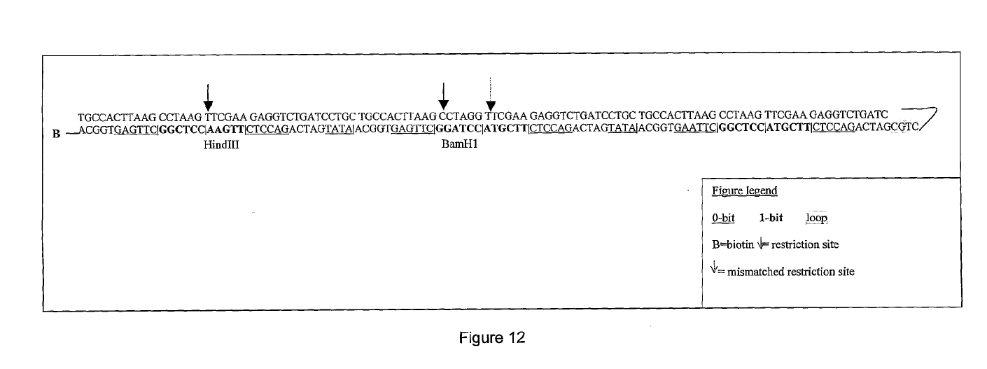

As shown in FIG. 12, the target with the unit sequence 0110 creates recognition sites for both BamH1 and HindIII at the 2^{nd} position in the 2^{nd} target copy and the 3^{rd} position in the 3^{rd} target respectively. The other potential restriction sites in the concatemer is inactivated as described for the target 0100 and 0010.

### Results

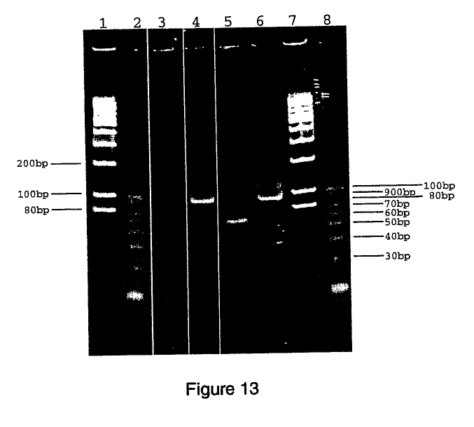

Target 0100 in all the experiments a band is visible on the gel at around the 60 bp marker (see lane 3, FIG. 13).

For target 0010, in all the experiments, a band is visible on the gel at around the 100 bp marker when digested with HindIII. However, in addition to the expected 96 bp band to other bands appear on the gel at around 50 and 40 bps. (see lane 4, FIG. 13). These results suggest that the HindIII digestion is not completely inhibited by a one basepair mismatch. If partial digestion occurs in the 2^{nd} target copy the expected molecules would be 96, 54 and 38. Digestion in the 1^{st} target copy should yield two bands of 38 and 16 basepairs. The fact that we do not see these bands on the gel might indicate that the restriction site is too close to the end of the molecule to cleave to any detectable degree. However, the fact that detection of the 54 and 38 basepair bands is found, indicates that a double-stranded DNA is formed during the rollback process.

For target 0110, in all the experiments, the same restriction pattern is seen when cut with BamH1 and HindIII as seen for Target 0100 and 0010 respectively (see lanes 5 and 6, FIG. 13).

The content of all the publications referred to herein are incorporated herein by reference.

I claim:

1. A method for analysing a target polynucleotide having distinct units of nucleic acid sequence, comprising:
(i) forming a first polynucleotide which is a concatemer having multiple repeating target polynucleotide sequences; and
(ii) hybridising a portion of the first polynucleotide to a second polynucleotide such that the portion hybridised, or the portion not hybridised, corresponds to at least a sequence unit on the target, and determining the sequence unit on the target.

2. (canceled)

3. The method according to claim 1, wherein the concatemer is formed by circularising the target polynucleotide and carrying out a polymerase reaction using the circular target polynucleotide as the template.

4. The method according to claim 3, wherein the circular target polynucleotide comprises an additional nucleic acid sequence.

5. The method according to claim 3, wherein the target polynucleotide is circularised by hybridising the target polynucleotide to the second polynucleotide, wherein 5′ and 3′ regions of the target polynucleotide hybridise to the second polynucleotide such that the 5′ and 3′ ends are in proximity, and ligating the 5′ and 3′ ends.

6. The method according to claim 3, wherein the target polynucleotide is circularised by hybridising an oligonucleotide to the target polynucleotide, wherein the oligonucleotide is complementary to both 5′ and 3′ regions of the target, such that the 5′ and 3′ ends are in proximity, and ligating the 5′ and 3′ ends.

7. The method according to claim 6, wherein the oligonucleotide is removed by an exonucleose prior to the polymerase reaction.

8. (canceled)

9. The method according to claim 1, wherein the units of nucleic acid sequence on the target polynucleotide are of either a first defined sequence or a second defined sequence.

10. The method according to claim 1, wherein each unit on the target polynucleotide comprises at least 4 nucleotides, and wherein the first sequence differs from the second sequence by two nucleotides.

11. (canceled)

12. The method according to claim 1, wherein the target polynucleotide comprises at least 40, units of nucleic acid sequence.

13. The method according to claim 1, wherein the sequence units on the target are unique to that unit position.

14. The method according to claim 13, wherein the second polynucleotide is designed to hybridise to each target polynucleotide of the first polynucleotide, but not to a predetermined sequence unit on each target, such that, after hybridisation, there is one sequence unit within one or more target polynucleotides that is not hybridised.

15. The method according to claim 14, wherein the sequence unit in the one or more target polynucleotides that is not hybridised, is determined.

16. A method for the formation of a double-stranded polynucleotide, comprising:
(i) forming a first polynucleotide; and
(ii) carrying out a rolling circle amplification reaction from a primer molecule attached to the first polynucleotide, wherein the amplification reaction utilises a circular polynucleotide molecule having a sequence that is at least partially complementary to repeating units of the first polynucleotide.

17. The method according to claim 16, wherein the circular polynucleotide is circularised by hybridising a single stranded polynucleotide to the first polynucleotide, wherein the 5′ and 3′ regions of the single stranded polynucleotide hybridise to the first polynucleotide such that the 5′ and 3′ ends are in proximity and ligating the 5′ and 3′ ends.

18. The method according to claim 16, wherein the circular polynucleotide is circularised by hybridising an oligonucleotide to the single stranded form of the polynucleotide, wherein the oligonucleotide is complementary to both 5′ and 3′ regions of the polynucleotide such that the 5′ and 3′ ends are in proximity, and ligating the 5′ and 3′ ends.

19. A method for the conversion of a target polynucleotide having distinct units of nucleic acid sequence into a polynucleotide having nucleic acid sequences separating one or more of the distinct nucleic acid sequence units, comprising:
(i) forming a second polynucleotide having defined first and second sequence units in a predetermined order; and
(ii) forming on the second polynucleotide, a first polynucleotide made up of a series of target polynucleotides, wherein the order of the first and second sequence units permits the specific interaction between the first and second polynucleotides, such that distinct units of nucleic acid sequence on the second polynucleotide can be distinguished from other distinct units by the extent of interaction.

20. The method according to claim 19, further comprising determining the sequence and/or order of the distinct sequence units on the basis of the interaction to thereby determine one or more specific sequence units on the target polynucleotide.

21. The method according to claim 19, wherein step (ii) is carried out by hybridisation between the first and second polynucleotides, wherein the first polynucleotide comprises sequence units which interact with the first sequence units of the second polynucleotide, but not to the second sequence units, and wherein the order of the first and second sequence units is such that the order of non-hybridised sequence units on the first polynucleotide represents a defined order of sequence units on the target polynucleotide.

22. The method according to claim 19, wherein the second polynucleotide is formed by the rolling-circle polymerase reaction.
